(19) United States
(12) Patent Application Publication
(10) Pub. No.: US 2014/0323593 A1
(43) Pub. Date: Oct. 30, 2014

(54) METHODS AND APPARATUS FOR THE CONTROL OF ADENOSINE TRIPHOSPHATE SYNTHASE ACTIVITY WITHIN LIVING ORGANISMS, AND CONDITIONING OF WATER-BASED FLUIDS AND SUBSTANCES USING MAGNETIC FIELD EXPOSURES OR THEIR WITHDRAWAL

(71) Applicants: Paul Héroux, Longueuil (CA); Ying Li, Longueuil (CA)

(72) Inventors: Paul Héroux, Longueuil (CA); Ying Li, Longueuil (CA)

(21) Appl. No.: 13/871,894

(22) Filed: Apr. 26, 2013

(51) Int. Cl. A61N 2/02; A61N 2/00; A61K 41/00

(52) U.S. Cl. CPC A61N 2/02; A61K 41/0052; A61N 2/004
USPC 514/789; 600/13

(57) ABSTRACT

The present invention relates to the control by magnetic fields (MF) of the activity of the mitochondrial enzyme adenosine triphosphate synthase (ATPS). Elimination of alternating and static MFs allows ATPS activity amplification (ATPSA), while a variety of MF frequency-intensity combinations interfere to various degrees with ATPS function (ATPSI). Control of ATPS, which is the endpoint of the mitochondrial oxidative phosphorylation system, allows alteration of intracellular adenosine triphosphate (ATP) levels, a molecule critical to the function of living organisms. Changes in ATP levels may trigger various events in cells, and prolonged ATPSI can lead to cell death. Specific combinations of MF frequencies and intensities maximize ATPSI for cancer cell killing and other health related applications.

This application follows Provisional Application for Patent No. 61/642,214, filed on May 3, 2012. It contains no new matter relative the version filed 2013 Apr. 26, only spacing changes.

### TECHNICAL FIELD

Health

### BACKGROUND ART

### Citation List

### US Patent Literature

Group A
2003/0026781 A1 Anderson et al., 2003
2010/0190845 Juan et al., 2010
U.S. Pat. No. 6,376,211 B1 Little I I et al., 2002
2007/0203083 Mootha et al., 2007
2011/0110914 A1 Narain et al., 2011
2003/0086929 A1 Tso et al., 2003

Group B
U.S. Pat. No. 6,238,899 B1 Blackman, 2001
U.S. Pat. No. 6,210,317 B1 Bonlie, 2001
U.S. Pat. No. 4,665,898 Costa and Hofmann, 1987
2002/0103515 A1 Davey and Williams III, 2002
U.S. Pat. No. 4,622,952 Gordon, 1986
U.S. Pat. No. 5,045,050 Liboff et al., 1991
U.S. Pat. No. 5,211,622 Liboff et al., 1993
U.S. Pat. No. 5,450,859 Litovitz, 1995
U.S. Pat. No. 6,263,878 B1 Litovitz, 2001
U.S. Pat. No. 6,856,839 B2 Litovitz, 2005
U.S. Pat. No. 7,587,230 B2 Litovitz, 2009
U.S. Pat. No. 4,815,448 Mills, 1989
2010/0016651 A1 Sivo, 2010
2010/0197993 A1 Vasista, 2010

Group C
U.S. Pat. No. 6,602,411 B1 Aida et al., 2003
2009/0071889 A1 Arenas et al., 2009
U.S. Pat. No. 4,146,479 Brown, 1979
U.S. Pat. No. 5,296,141 Ellison, 1994
U.S. Pat. No. 6,797,159 B2 Hatton, 2004
2005/0126974 A1 Naito, 2005
U.S. Pat. No. 6,171,504 B1 Patterson, 2001
2009/0306456 A1 Pilla et al., 2009.
U.S. Pat. No. 5,288,401 Rodriguez, 1994
U.S. Pat. No. 3,951,807 Sanderson, 1976
2009/0242407 A1 Shiga, 2009
2008/0277352 A1 White, 2008
2009/0261028 A1 Yamasaki, 2009
U.S. Pat. No. 7,943,043 B2 Yamasaki, 2011

### SUMMARY OF INVENTION

We provide methods and apparatus to control the activity of the enzyme ATPS within living organisms, and to condition water-based fluids to obtain the same effect. ATPS molecules are imbedded in the inner membrane of the numerous mitochondria contained within individual human and mammalian cells, as well as in the cell walls of bacteria.

The apparatus is a MF control system exposing living organisms to specific MF conditions acting as an amplifier (ATPSA) or inhibitor (ATPSI) of ATPS activity. Exposure of water-based fluids to similar specific MF conditions induces ATPSA or ATPSI in organisms that are subsequently immersed in these fluids.

Because relatively non-toxic MFs at low frequencies penetrate living organisms practically unaltered, it is possible by external circuit control to achieve uniform and relatively rapid changes in the synthetic activity of ATPS within organisms, and also to focus ATPSA or ATPSI on specific locations within the body, using MF gradients.

Water-based fluids treated under ATPSA or ATPSI exposure conditions can impart the corresponding effects to organisms interfacing with the treated fluid, leading to practical applications using properly conditioned imbibed or injected fluids.

The method for amplification of ATPS activity, ATPSA, is the most simple, consisting of the elimination of both technological MFs (including static, Extra-Low Frequency, Voice-Frequency, Very-Low Frequency, Low Frequency, Radio-Frequency and Micro-Wave MFs), as well as Earth's static field to levels below 10 nT, for complete effect.

The method for disrupting ATPS activity, ATPSI, applies specific MFs, and is effective over a variety of frequencies and MF intensities. This range of frequencies and intensities is observed because ATPSI essentially disrupts a delicate molecular property of water molecules (proton tunnelling conductance), which in turn perturbs ATPS function. The ATPSI MFs must be maintained for many hours (for example, 3 to 4 hours for an Extra-Low-Frequency signal) to obtain the corresponding level of ATPS activity. Conversely, many hours without MF are necessary for restoration of a previous ATPS activity level.

We have observed that relatively rapid changes in ATPS efficiency, inducible by MFs, can be used to trigger apoptosis in some cancer cells, and that sustained application of certain MF frequency and intensity combinations can be used to starve cancer cells of adenosine triphosphate (ATP), leading to their death. “Tuning” to specific frequency and MF intensity combinations is needed in the strong ATPSI of cancer cell killing applications. However, a number of frequency-intensity combinations have been observed to be effective, and simultaneous application of many such combinations may provide the strongest ATPSI.

Cancer cells, because of their active and altered metabolism, are particularly sensitive to ATPSI, which is an effective method of cancer cell killing, particularly under the low oxygen conditions found in tumours (82% of readings in tumours are less than 0.33% oxygen) and in the body tissues generally (1 to 6% oxygen). Specific cancer cell types may vary in their dependence on oxidative phosphorylation, which can be altered by ATPSI over long periods. In such a case, various levels of ATPSI or ATPSA over a few weeks of exposure can be used to alter the metabolic preferences of such tumors, enhancing the efficiency of the acute ATPSI cell killing procedure. As well, weakly toxic pharmacological metabolic inhibitors, of glycolysis for example, can be combined with ATPSI for stronger therapeutic action. Finally, conventional chemotherapeutic agents can be combined with metabolic inhibitors and ATPSI in cancer therapy.

Important advantages of the ATPSI treatment are the ability of MFs to uniformly penetrate living organisms with negligible attenuation, to be applicable either to the whole body by a uniform field or in focused form using MF gradients, to leave behind no metabolites, resulting in inherently low toxicity, and to suppress or enhance ATPS activity rapidly through electrical control of the MF. The methods we describe could be used in acute treatment or in chronic treatment to prevent cancer recurrence. The methods and apparatus we describe can be used to eliminate tumours from living tissue, alone or in conjunction with a chemotherapeutic cancer agent. Numerous other applications, which involve metabolic control, can be envisioned.

We have also observed that water-based fluids, when exposed to ATPSA or ATPSI conditions, could retain their ATPSA or ATPSI properties for some hours. Therefore, solutions exposed to ATPSA or ATPSI conditions can be imbibed or injected into living organisms, and exert their specific effects, even if the organism itself has never been exposed to ATPSA or ATPSI.

### TECHNICAL BACKGROUND OF THE INVENTION

Importance of ATP

Adenosine-5′-triphosphate (ATP) is a cellular coenzyme widely involved in cellular energy transfers. During enzyme-catalyzed hydrolysis of ATP or phosphorylation by ATP, the free energy of the phosphate bond is harnessed by living systems to do work. Enzymes called kinases use ATP as a substrate in signal transduction pathways to phosphorylate proteins and lipids, and adenylate cyclase also uses ATP to produce the second messenger cyclic AMP. ATP powers enzymes, biosynthesis of structural proteins, motility and cell division. ATP is incorporated into nucleic acids by polymerases in the processes of DNA replication and transcription.

ATP can be produced by three distinct cellular processes: in the cytosol by (1) glycolysis, and, in mitochondria, by (2) the citric acid cycle or oxidative phosphorylation, and (3) beta-oxidation. ATP is continuously recycled, and the major production takes place in mitochondria, which make up nearly 25% of the total volume of a typical cell. ATP production by glycolysis is less efficient than by mitochondrial oxidative phosphorylation, where it is produced by ATP synthase (ATPS) from inorganic phosphate and adenosine diphosphate (ADP) or adenosine monophosphate (AMP). The transformation of glucose to carbon dioxide is known as cellular respiration, and can produce about 30 molecules of ATP from a single molecule of glucose. In mitochondria, an electron transport chain pumps protons out of the mitochondrial matrix and into the intermembrane space, creating a proton motive force, a pH gradient and an electric potential across the inner mitochondrial membrane. Flow of protons down this potential gradient provides the driving force for ATP synthesis by ATPS. Most ATP synthesized in mitochondria is actively transported for use in the cytosol.

Importance of ATPS

ATPS (EC 3.6.3.14) is present in all living organisms, and is located in the membranes of mitochondria, bacteria, and chloroplast thylakoids, as well as on the surfaces of various cell types, including endothelial cells, keratinocytes, and adipocytes. In mammalian cells, ATPS is a critical enzyme that provides the bulk of cellular energy through the synthesis of ATP. ATPS consists of two parts, the F_{O} portion within the mitochondrial inner membrane, and the F_{I} portion outside the membrane, protruding in the mitochondrial matrix. F_{O} (written as a subscript letter “o”, not “zero”) derives its name from being the oligomycin-binding fraction. Oligomycin, an antibiotic, inhibits the F_{O} unit of ATPS, and we consider it the closest molecular equivalent of MF action on biological materials.

The protons generated by the electron transport chain of mitochondria (Complexes I to IV) accumulate in the inter-membrane space, and are electrostatically driven across the inner membrane through the F_{O} region of ATPS, which includes hydrophilic proton channels. A rotary mechanical force resulting from proton motion is used by ATPS F_{I} to synthesize ATP. Under the right conditions, the enzyme reaction can also be carried out in reverse, with ATP hydrolysis driving the pumping of protons across the membrane. In mammalian cells, the ATPS of mitochondria shows large amounts of similarity to the vacuolar ATPS used to generate a proton gradient and pH values as low as 1 inside cellular digestive compartments, endosomes and lysosomes, at the expense of ATP.

A similar reversible role is present in bacteria. Fermenting bacteria that do not have an electron transporting chain hydrolyze ATP to generate a proton gradient, which they use for flagella and transport of nutrients into the cell. But in respiring bacteria, ATPS in general runs in the opposite direction, creating ATP using the proton-motive force created by the electron transport chain (oxidative phosphorylation).

A person consumes its own weight (75 kg) in ATP every day, yet contains only 50 g of ATP, which means that ATP molecules are recycled 1500 times per day. ATP supplies therefore last roughly one minute, and any change in its regeneration rate will be rapidly felt unless rapid adjustments in metabolism are made.

The ratio between ATP and AMP is sensed with great accuracy by the enzyme adenosine monophosphate activated protein kinase (AMPK), which monitors the level of energy available to the cell, and controls the metabolic pathways that produce and consume ATP. When mitochondrial ATPS is inhibited by MFs, the perturbations in ATP levels activate AMPK, because healthy cells must maintain a high level of phosphorylation capacity (ATP:ADP≈10) to function well. AMPK is a sensitive regulator that switches “on” catabolic pathways, and “off” many ATP-consuming processes, both acutely and chronically, through gene expression.

Inhibition of ATPS

Inhibition of ATPS by Drugs

Because of the strategic importance of ATPS, inventories of molecular means for controlling it have been made (Hong and Pedersen, 2008). There are many agents that inhibit ATPS F_{O}F_{I} and ATPS F_{I}, but fewer that inhibit ATPS F_{O} specifically. Still, the list of ATPS F_{O} inhibitors is fairly long: leucinostatins, diethylstilbestrol, genistein, the polyketide macrolides (apoptolidin, cytovaricin, oligomycin, ossamycin, and venturicidin), rhodamine 6G, tetracaine, dicyclohexyl-N-acylurea, chlorpromazine, trifluoroperazine, N,N′-dicyclohexylcarbodiimide, N-(2,2,6,6-tetramethylpeperidyl-1-oxyl)-N-(cyclohexyl)carbodiimide, Woodward's reagent K, tetranitromethane, 1,5-difluoro-2,4-dinitrobenzene, N-Ethylmaleimide, p-Chloromercuribenzoic acid, p-Chloromercuribenzene sulfonate, Mersalyl, 2,2-Dithiobispyridine, Diethyl pyrocarbonate, R207910, Dihydrostreptomycin, 4,4-Dichlorodiphenyltrichloroethane (DDT), zinc and Bz-423 (a lupus drug). A few of those ATPS inhibiting molecules, oligomycin and apoptolidin, have been studied in more detail for their potential as anti-cancer drugs.

Inhibition of ATPS by Magnetic Fields

The structure of ATPS is documented in detail as a rotating motor-generator structure activated by the trickle of high-density protons from the mitochondrial inter-membrane space into its matrix. Proton diffusion along the 15 nm thick inter-membrane space does not limit their transit time (1-2 μs). Protons enter the F_{O} of ATPS into an entry half-channel made of four hydrophilic α-helices, to reach a rotating helix. After rotation, protons flow out through a similar exit half-channel. The rotation is used by the F_{I} segment of ATPS to produce ATP. Power-frequency MFs influence the flow of protons through the channels of ATPS. These hydrophilic pockets provide a high density of hydrogen bonds, while the mitochondrial inter-membrane space feeds a high-density of protons. The high-density protons (pH 1) are driven through the channels by a 180 kV/cm electric field across the inner membrane. It was documented by electrical dissipation factor (ωRC, also known in electrical engineering as tg δ) and optical measurements (the dimerization of dilute rhodamine 6G solutions) that alternating MFs in the range 25 nT-879 μT alter the arrangement of water molecules, particularly under high concentrations of hydrogen bonds and protons (Semikhina at al, 1988), increasing proton impedance (decreased soliton tunnelling) through hydrophilic channels such as those in ATPS. This tunnelling of protons exploited by ATPS has also been observed as double wells in neutron Compton scattering studies performed on water-filled nanotubes. This nanotube confinement of water molecules on scales of 2 nm or less (Reiter et al, 2011) is of the same character as the water channels contained within ATPS's 7.5 nm (diameter) rotor (Miller et al., 2001). For reference, electrons in a scanning tunnelling microscope flow in a channel 0.5 nm in diameter. Another link between MFs and ATPS is provided by the inhibitory action of rhodamine 6G, the dye monitored in Semikhina's MF experiments, on the F_{O} segment of ATPS.

In summary, application of MFs reduces proton flow through ATPS.

Experiments (Semikhina et al, 1988) showed a progressive inception of MF effects on water over 5 hours, and dissipation over 2 hours, after the field is turned off. The first time interval should be close to the MF exposure time needed to induce full changes in the water structures within ATPS, and the enzyme's efficiency. The effects on water are absent above 40-50° C., as water structure changes (Semikhina and Kiselev, 1981).

We verified the effects of MFs on ATPS by a variety of techniques.

First, we observed that 5 different cancer cell lines representative of the most common human cancers display a reduction in their chromosome numbers when exposed to 60-Hz MFs (Appendix 1). Reductions in chromosome numbers result from metabolic restriction (Li et al., 2012), which can be caused by impaired ATPS function. The administration of the ATPS F_{O} inhibitor oligomycin induces similar reductions in chromosome numbers in the same cells.

Second, when MFs are applied to cancer cells, reductions in chromosome numbers are observed starting at the threshold (25 nT), and extending over two orders of magnitude, as predicted by Russian physicists for the effects of MFs on water. More detailed studies have shown that the effect occurs over a wide variety of amplitudes and frequencies, relevant to environmental Extra-Low-Frequency (ELF) MFs.

Observations are thus compatible with a disruption in water structure with little sensitivity to MF intensity, or to the particularities of specific cell type metabolism: the knockout of an important biological enzyme by changes in water structure.

Third, we monitored changes in K562 culture histograms of cell size induced by 60-Hz 0.4 μT MFs and by 5 other anti-oxidants. We fbund that the histogram measured for MFs uniquely matched the characteristic of the specific ATPS F_{O} inhibitor oligomycin A (3.2 nM). Oligomycin in known to inhibit ATPS by binding to its F_{O} segment, which contains the water channel used by protons. The F_{O} S subunit is also designated “oligomycin sensitivity conferral protein”.

Fourth, Russian physicists (Semikhina and Kiselev, 1981) observed resonances for water at specific frequency-amplitude combinations that are broadened by the presence of even small levels of impurities. They detected one maximum at 156.2-Hz and 15.45 μT for 7° C. pure water (Semikhina et al, 1988). In our experiments compiling average chromosome losses over frequency in anoxic K562 (5% carbon dioxide and 37° C.) 6-day tests at 1 μT, a wide resonance was measured as follows: −3.6±0.79 at 50-Hz, −9.36±1.06 at 60-Hz, −12.71±1.82 at 120-Hz and −9.8±1.31 at 155-Hz. A polynomial fit on this data predicts maximum MF effect on ATPS at 113 Hz for 1 μT. The ATPS resonance at 1 MT we documented is indeed wider than that reported by Semikhina for pure water.

Fifth, if MFs inhibit ATPS, they should also activate AMPK. The MF>ATPS>AMPK pathway was investigated using metformin and resistin. Metformin is a diabetes drug that activates AMPK, leading to reduced glucose production in the liver, and reduced insulin resistance in muscle. It is an attractive anti-aging drug that usually causes weight and appetite loss. Resistin, a product of the RSTN gene, is a 9.9 kDa protein containing 93 amino acid residues which, at 20 ng/ml or more, inhibits AMPK. It interferes with phosphorylation of Akt (serine/threonine protein kinase), active in multiple cellular processes such as glucose metabolism, cell proliferation, apoptosis, transcription and cell migration.

Metformin (0.01 mg/l) and resistin (40 ng/l) alone for 3 days induce average reductions in chromosome numbers of 9 and 10 respectively, in the leukemic cell K562. When 1 μT is added to metformin, even larger losses are observed (9 becomes 11±0.34). When 1 μT is added to resistin, the losses of resistin reduce from 10 to 4±0.46, also less than the losses of 1 μT alone, at 7.5. The conclusion is that MFs enhance the action of metformin, but neutralize the effect of resistin, again suggesting a connection between MFs and ATPS.

Sixth, Russian physicists stated (Semikhina and Kiselev, 1981) that when alternating MFs were kept to values below 25 nT, an influence of static MFs on water could be detected. Specifically, removing the static MF acted on water variables (dissipation factor and optical measurements) in a direction opposite to the application of ELF MFs larger than 25 nT. Thus, substantial elimination of both ELF and static MFs would allow water to optimize its molecular arrangement, which we surmised would make water more permeable to protons, and improve ATPS efficiency.

The influence of the static MF was investigated by observing K562 cells transferred from a steel shield that eliminated ELF MFs (to less than 5 nT), but had a static field of 74 μT, to a second shield that attenuated both the ELF MF (less than 5 nT) and the static field to 3 μT. An acrylic cylinder 5.7 cm in internal diameter with 0.38 cm wall and 38 cm long was covered by 10 layers of 0.4 mm Nickel-Iron-Molybdenum alloy (NIM, ASTM A753 Type 4), each spaced by 1.6 mm. After 4 days, cell numbers in the NIM shield were increased by a factor of 2.05±0.13 (standard deviation) over cells kept in the steel shield, indicating enhanced metabolism. The effect is persistent over time. Consequently, our observations on cellular metabolism fall in line with the predictions of Semikhina and Kiselev for proton tunnelling as influenced by both static and ELF fields.

These observations suggested to us that ATPS proton flow could be controlled by applying MFs to living organisms, thus allowing direct action on the core of human metabolism. This control over ATPS and ATP production creates the opportunity for opposite states which we call ATPSA (amplification of ATPS activity), brought about by very small MFs and ATPSI (inhibition of ATP production) through a variety of specific MF exposures. Various MF environments thus allow modulation of ATPS activity.

Advantages of Magnetic Fields Over Drugs

Although MFs can control ATPS, there are a number of drugs and chemicals that inhibit either the F_{I} or the F_{O} segments of ATPS, producing similar effects, as discussed previously. To understand the advantages of MFs over drugs, one must consider the problems associated with the clinical deployment of drugs that have been found effective in vitro. Such problems are commonly abbreviated as “ADMET”. This acronym signifies that drugs typically have specific difficulties in terms of their Absorption, Distribution, Metabolism, Excretion and Toxicity.

Absorption: drugs reach their target sites according to their solubility in water and fat, their partition coefficient, their ability to ionize in water, and their molecular size. As a result, some molecular species that are therapeutically ideal may have a poor ability to reach their target.

Distribution: providing effective drug concentrations to targets may mean that higher and unwanted concentrations exist in other compartments of the body, because distribution varies according to micro-anatomy and limiting membrane characteristics.

Metabolism: if drugs are to be administered either by mouth or intravenously, they need to resist enzymes and conditions in the digestive system or in the blood. Even if they can survive these environments, drugs can be metabolized from their primitive form to ultimate forms that may be toxic.

Excretion: as drugs are excreted, they may concentrate in the liver or kidneys, in particular, at levels that are toxic and life-threatening to the whole organism.

Toxicity: even after the refinements in molecular structure and dosage regimens that is typical of pharmacological development, the most successful drugs usually show side-effects, toxicities that could not be entirely avoided.

ADMET considerations have prevented many useful molecules from ever reaching medical applications.

In terms of absorption and distribution, MFs are the ultimate vector, because they can be generated uniformly over large volumes (using solenoid coils), and because they penetrate the body instantly, uniformly and essentially without change or attenuation, at least at lower frequencies.

Problems of metabolism are attenuated or eliminated, since MFs are not molecules, and cannot be modified by chemical or enzymatic reactions.

In terms of excretion. MFs offer great advantages over drugs in providing easily controllable kinetics. With drugs, there is often little control over the rise time of concentration at the target site, as well as on its rate of elimination, which is determined by enzymatic bio-transformation, fluid transit to excretion compartments, and diffusion rates in organs such as liver and kidney. By electronic switching, MFs can be applied or eliminated instantly, and the effects on ATPS are only limited by the speed of water's re-structuring reactions, which are relatively rapid compared to the distribution and elimination times of many drugs, particularly in creating ATP surges.

Toxicity is reduced for drugs according to their selectivity, that is, the concentration of effects on the target, as opposed to all other sites. The action of MFs on ATPS is highly specific to mitochondrial ATPS F_{O}, because of the need for the intersection of two conditions for effectiveness: a high density of hydrogen bonds (supplied by the hydrophilic channel of ATPS) and high density of protons, found in mitochondria and in cellular digestive organs, endosomes and lysosomes. MFs have a huge specificity advantage over chemotherapeutics such as alkylating agents, that act on tumors by creating wide-spread molecular damage.

The polyketide macrolides oligomycin and apoptolidin have been confirmed as specific inhibitors of ATPS F_{O}, and are expected to exert physiological effect similar to MFs. The closest molecular equivalent to MFs, the drug oligomycin, would be expected to be more toxic than MFs because, beyond being a reversible inhibitor of mitochondrial ATPS F_{O}, it also inhibits the Na⁺, K⁺-ATPase that is present in animal plasma membranes, some drug-translocating ATPases that confer drug-resistance to mammalian and yeast cells, and is further suppressive for human B cell activation. As a bonus. MFs do not leave any molecular derivatives behind when they are turned off.

### ATP and Cell Survival

### Mitochondrial Metabolism

The ATP generation function of mitochondria occurs through an electron transport chain (Complexes I, II, III, IV) driven by metabolism that creates a concentration of protons in the mitochondrial inter-membrane space. The protons are used by ATPS (also known as Complex V) to generate ATP. Electron transport is coupled to proton consumption by ATPS. The higher the proton concentration in the inter-membrane space, the lower the electron flow, and vice versa. Under resting conditions, the demand for new ATP is limited, and so is the flow of protons through ATPS. When ATP consumption increases, such as during vigorous muscle activity, protons pour through ATPS, regenerating the ATP pool. The rate of electron transport is usually measured by assaying the rate of oxygen consumption and is referred to as the cellular respiration rate. There are several well-known drugs and toxins that, like MFs, inhibit oxidative phosphorylation, as seen in Table 1 below.

TABLE 1
Inhibitors of Oxidative Phosphorylation

| Molecule | Function | Site of Action |
|---|---|---|
| Rotenone, Amytal | Electron transport | Complex I |
| | inhibitors | |
| Antimycin A | Electron transport | Complex III |
| | inhibitor | |
| Cyanide, Carbon | Electron transport | Complex IV |
| Monoxide, Azide | inhibitors | |
| 2,4,-dinitrophenol, | Uncoupling | Transmembrane |
| Pentachlorophenol | agents | proton carrier |
| Oligomycin | Inhibits ATPS | F_{O} of ATPS |

Although any one of these toxins inhibits only one enzyme in the electron transport chain, inhibition of any step in this process will halt ATP synthesis. For example, as oligomycin inhibits ATPS, protons cannot pass back into the mitochondrial matrix. Accumulated protons increase the membrane voltage, which proton pumps are then unable to overcome. NADH is no longer oxidized, and the citric acid cycle ceases to operate, because the concentration of NAD⁺ falls below the concentration that these enzymes can use.

Uncoupling agents have been found that associate with protons, pass through the membrane with the bound proton, and dissociate the proton on the interior of the mitochondrion. These agents cause maximum respiration rates (oxygen consumption) but generate no ATP, since the translocated protons do not return to the matrix through ATPS. An example of an uncoupling agent is rutamycin A, a macrolide antibiotic obtained from Streptomyces rutgersensis. It is used clinically as an antifungal agent, but like other oligomycins, has not been used internally because of its demonstrated toxicity to mice and HeLa cells.

### ATP Starvation

Although it is rather intuitive that sufficient reductions of cellular ATP levels will lead to the death of any type of cell, as homeostasis cannot be sustained, metabolic particularities specific to cancer cells make them more vulnerable than normal cells. At four moments of a normal cell's cycle, ATP needs to increase by as much as 4-fold (Edwards and Lloyd, 1977), depending on current metabolism (glycolysis or oxidative phosphorylation). Glucose metabolizing cells have larger changes in ATP needs than cells dependent on oxidative phosphorylation.

One of the earliest observations on cancer cells is that in the presence of oxygen, they do not reduce their glucose metabolism (Warburg, 1956) as normal cells do. This metabolic de-regulation of cancer cells increases their peak ATP needs to a wider dynamic range than normal cells, which can only be met by both glycolysis and oxidative phosphorylation together (van der Windt et al., 2012). ATPS inhibitors like oligomycin and MFs impose a ceiling on the amount of ATP available in the cell, reducing its spare respiratory capacity, as it goes through a variety of energy-intensive cellular processes.

There is also a separate or complementary understanding of the cell death mechanism under ATP starvation. ATPSI increases the mitochondrial inner membrane potential (ΔΨm) by impairing proton flux. Sustained ATPSI by a continuously applied MF should lead to ΔΨm hyperpolarisation. Such a high proton motive force in “state 4” is dangerous for the cell, due to an increase in the probability of superoxide radical formation, leading the cell into apoptosis (Korshunov et al., 1997; Skulachev, 1996).

### ATP Synthesis Transitions

Beyond ATP starvation, a second cell killing strategy relates to modulation of ATPS activity by producing rapid changes both in ATP levels and in ΔΨm that could induce cell death through apoptosis. Changes in both alternating and static MFs together can produce steep ATP rises (reduced fields) and falls (increased fields).

Although ΔΨm increases are thought to be move effective than decreases (Samudio et al., 2009) in inducing apoptosis, water may respond more rapidly to MFs switching “off” (ΔΨm decreases) than turning “on” (ΔΨm increases), thereby maximizing ∂ΔΨm/∂t. Extreme and rapid changes in ATPS activity lead to a rapid shift in aerobic vs anaerobic balance within the cell, which is key to achieving maximum killing effect in cancer cells (Salomon et al., 2000). Another important aspect of MF therapy is that, as we shall see below, oxidative to glycolytic balance of cellular metabolism can be shifted over time by sustained MF exposures.

### Magnetic ATPS Inhibition and Cancer

### Low MF Toxicity

That normal cells are not acutely vulnerable to MFs is demonstrated by the public's chronic exposure experience. In the last century, where human societies became highly electrified, a wide variety of MF exposures have been considered safe. For example, the American Conference of Governmental Industrial Hygienists quotes an 8 hour per day safe Threshold Limit Value for occupational exposures at 60-Hz as 1000 μT.

### Magnetic Field Specificity to Cancer Cells

Untransformed (non-cancerous) cells have low ATP requirements that can be met with basal mitochondrial activity (Salomon et al., 2000). The unique metabolic profile of cancers (aerobic glycolysis) means that several human cancers have high ΔΨm compared to normal cells (Bonnet et al., 2007). In this case, a logical strategy is to increase this potential even further, into the damage zone, by ATPSI.

If some tumors or tumor components do not have a high ΔΨm, the challenge can be met in practical cancer therapies by using a sequence of longer-term MF applications (compared to acute killing over days) to shift their metabolism, or by using complementary pharmacological strategies of low toxicity that inhibit other metabolic pathways, as we shall explain below.

An important aspect of the specificity of action of MFs on cancer cells is that in spite of the inhibitory effect of MFs on ATPS, large amounts of available oxygen can overwhelm the inhibition. It is for this reason that we have observed in our experiments that the intense ATPSI required for cancer cell killing is more effective as the level of oxygen decreases in the cell culture. Since it is reported that 82% of oxygen readings in tumors are less than 0.33%, while body tissues are generally between 1 to 6%, MF therapy could be expected to be particularly effective in the poorly perfused parts of tumors that are most remote from pharmacologic interventions, and where stem cancer cells are possibly hiding within niche structures.

As we consider oligomycin the closest molecular equivalent to MF action, its properties are of particular interest. A critical property of the macrolides is their specificity to cancer cells, as shown by their ability to selectively kill E1A and E1A/E1B19K (adenovirus genes) transformed rat glial cells, while not killing untransformed glial cells: these drugs selectively sensitize cancer cells to apoptosis induction (Salomon et al, 2001). A similar specificity is expected for MFs.

Killing Cancer Cells with Oligomycin

Oligomycins A, B, and C are a family of macrolide antibiotics produced by Streptomyces diastatochromogenes. They have antifungal activity, but have not been clinically useful because of their toxicity demonstrated in mice. Other macrolide antibiotics, however, are used clinically. Erythromycin, for example, has an antimicrobial spectrum wider than penicillin. The attention of biochemists was drawn to oligomycin in spite of the toxicity because of its ability to inhibit oxidative phosphorylation in mitochondria (Lardy et al., 1965), and because it was shown to be among the top 0.1% most cell line selective cytotoxic agents of 37,000 molecules tested against the battery of 60 cancer cell lines maintained by the US National Cancer Institute (NCI-60). This means that oligomycin was lethal at extremely small doses in some cell lines, while being much less effective in others. In fact, variations in the IC₅₀ (the concentration inhibiting proliferation rate by 50%) varied by more than 3 orders of magnitude between cell lines, with a mean of 300 nM. Evaluation of the cause behind these variations led to the conclusion that the mitochondrial apoptotic pathway could be triggered in response to a shift in balance between aerobic and anaerobic ATP biosynthesis. In other words, if the cells that were relatively insensitive to oligomycin could somehow have their metabolism shifted towards aerobic ATP biosynthesis, as measured by the suppression of genetic expression of hypoxia inducible factor 1α (HIF-1α), they would become sensitized to oligomycin, which would then become a general solution to cancer cell eradication. Even in conventional chemotherapy, it is recognized that mitochondria must be primed for optimal triggering of apoptosis (Reed, 2011). In one study (Salomon et al., 2000) the metabolic shift was achieved by adding oxamate, an inhibitor of lactate dehydrogenase with 2-deoxyglucose, which inhibits carbon flux through the glycolytic Embden-Meyerhofpathway, thereby shifting equilibrium towards aerobic ATP biosynthesis. Anti-oxidant metabolic restrictions that are either pharmacological or MF-based will trigger HIF-1α, a transcription factor regulating key glycolytic enzymes including aldolase, lactate dehydrogenase, pyruvate kinase (controls the aerobic status of cells), enolase, and phosphofructokinase (Semenza, 1999). These enzymes collectively mediate increased glycolytic generation of ATP and other intracellular metabolic adaptations to hypoxia that play a role in tumor progression, but that may also make cancer cells more vulnerable to ATPSI.

Killing Cancer Cells with Magnetic Fields

### Weak ATPSI

Our laboratory has detected ATPSI at all tested frequencies and at all MF intensities above 25 nT using measurements of karyotype contraction (see Appendix 1) in numerous cell lines: K562 and HEL (erythro-leukemias). MCF7 (breast), NCI-H460 (lung) and COLO 320DM (colon). In our experiments, ATPSI detected by karyotype contractions varied only weakly with field intensities between 25 nT and 5 μT, but probably cover the range of 10 nT to 879 μT. The full range of frequencies over which ATPSI occurs has not been documented yet.

### Strong ATPSI

While weak ATPSI can be obtained by diverse conditions of MF intensity, frequency and exposure, particular conditions of (1) frequency, (2) MF intensity and (3) duration of must be met to obtain the strongest ATPSI exposures for cancer cell killing. These conditions are loosely related by f/B=k, which means that optimal conditions are often obtained at lower field intensities (B) for lower frequencies (f). Specific “transition” frequencies, 86, 120 and 156.2 Hz, are particularly favourable for strong ATPSI, with optimal MF intensities of approximately 0.43, 0.707 and 0.742 μT respectively. Cell death occurs in 1-3 days, under conditions where the static MF is comparable to the geomagnetic field. We are still refining our techniques to find the exact conditions (the peaks are quite narrow over frequency and MF intensity) that are optimal for this application. The cell killing process can proceed through various stages where the cells display culture synchronization, necrosis or interrupted apoptosis. Killing has been confirmed in the two cell lines tested, K562 and MCF7, and optimal conditions do not seem to differ according to cell type. The most successful algorithms may involve the application of many frequencies simultaneously as well as a sequence of ATPSI or ATPSA steps to prime tumor cell metabolism for effective cancer cell killing.

MFs are potentially an entirely new type of strategies for eliminating cancers, since they are based not of alkylation or pharmacological inhibition, but on Quantum Electrodynamics.

### ATPS Amplification: Enhancing Metabolism

According to Russian physicists (Semikhina and Kiselev, 1981), proton tunnelling through water is impaired by ELF fields higher than 25 nT, but improved by counteracting the geomagnetic field. Thus, removing both alternating and static fields would be expected to increase the efficiency of ATPS function. Our observations on K562 cells reported earlier in this document confirm this influence of the static field, whose withdrawal allows water to optimize its tunnelling efficiency beyond what is achieved by removal of any alternating MFs. This implies that the oxidative phosphorylation process of mitochondria's ATPS can be substantially enhanced not only by eliminating alternating MFs, but also by reducing the Earth's natural MF (50 μT) or any static field, thereby providing a non-invasive and artificial method for metabolic enhancement in biological organisms.

### Magnetic ATPSA and ATPSI in Water-based Fluids and Substances

It was found in our laboratory (Appendix 1) that cells grown for 7 hours under identical incubator conditions fared differently according to whether the culture medium added at 0 hours originated from closed flasks exposed overnight to: Very Small MF (<4 nT at 60-Hz, 3 μT static), Incubator MF (2 to 2.7 μT at 60-Hz) or Inhibitory MF (0.62 μT at 120 Hz).

After the sealed flasks containing cell culture media (only) are exposed to their respective MFs overnight, cells are introduced into each flask, and incubated for 7 hours under incubator conditions (2 to 2.7 μT at 60-Hz). Measurements of cells numbers of each size are acquired at 0 hours, as well as at 7 hours for each flask, using a Millipore Scepter cell counter. There is an increase in the number of living cells observed under the Very Small MF condition, compared to the inhibitory condition, with the incubator condition rating in between. When unhealthy cells (originating from a culture with depleted medium) were used, the inhibitory MF had the effect of increasing the level of decay products (object diameters less than 10 μm) in the culture.

These results imply that MF action is effective in conditioning the cell culture medium, and has a lasting effect even after the MF is turned “off”, compatible with the results of Russian physicists on water (Semikhina et al, 1988). The memory effect described above on cell culture media is also observable as pH measurements in the cell culture medium, which turns slightly more acidic with increasing MF exposures. For example, at 60-Hz, there is a difference of −0.0722 pH units with a 95% confidence interval of 0.0266 between identical media exposed to Very Small MFs compared to 5 μT 60-Hz (Appendix 1). This was verified for a variety of cell media.

Because effects of the MF on water structure dissipate after a few hours, the implication is that ATPSA applied through fluids for even short periods can have a decisive effect on the evolution and survival of cells. The relatively long “magnetic memory” of water can thus be used to alter metabolism if conditioned fluids are administered to organisms exposed to different MF environments. Media exposed to Very Small MF can create a boost in metabolism through ATPSA that may be important under critical conditions such as blood injections in surgery or stroke patients. The applicability of the method needs to be investigated in detail, as ATP concentration increases may encourage or inhibit apoptosis depending on circumstances. It is also possible that imbibed ATPSA fluids or water-containing substances could have inotropic effects on sport performance.

There may also be applications for imprinting on fluids and water-containing substances the magnetic signature of ATPSI by exposing them for many hours to ATPSI-effective MFs.

### BRIEF DESCRIPTION OF DRAWINGS

FIG. 1

Whole body ATPSA setup providing low dc and ac MFs by neutralizing the static fields using a solenoid powered by a dc source and a magnetic shield attenuating to very low values the dc and ac MFs.

FIG. 2

Whole body ATPSA setup providing low dc and ac MFs by neutralizing the static fields using a pair of Helmholtz coils powered by a dc source and a magnetic shield attenuating to very low values the dc and ac MFs.

FIG. 3

Whole body ATPSA setup providing low dc and ac MFs by neutralizing the static and alternating fields using three orthogonal pairs of Helmholtz coils powered by a combined dc and ac source controlled by a triaxial MF sensor attenuating to very low values the dc and ac MFs.

FIG. 4

Whole body ATPSI setup providing neutralizing ambient dc and ac MFs using a solenoid powered by a dc source and a magnetic shield attenuating to very low values the dc and ac MFs. The injection of undisturbed ATPSI fields is either through the outside solenoid (A) or through an added solenoid placed inside (B) the magnetic shield.

FIG. 5

Whole body ATPSI setup providing neutralizing ambient dc and ac MFs using a pairs of Helmholtz coils powered by a dc source and a magnetic shield attenuating to very low values the dc and ac MFs. The injection of undisturbed ATPSI fields is either through the outside pairs of Helmholtz coils (A) or through an added pairs of Helmholtz coils placed inside (B) the magnetic shield.

FIG. 6

Whole body ATPSI setup providing neutralizing ambient dc and ac MFs using three orthogonal pairs of Helmholtz coils powered by a combined dc-ac source attenuating to very low values the dc and ac MFs. The injection of undisturbed ATPSI fields is through one pair of Helmholtz coils, while the MF feedback probe is compensated (offset) for the injected ATPSI signal.

FIG. 7

Focused ATPSI setup neutralizing ambient dc and ac MFs using three orthogonal pairs of Helmholtz coils powered by a combined dc-ac source attenuating to very low values the dc and ac MFs. The injection of the undisturbed ATPSI field generating ATPSI conditions at a single point is through pairs of Helmholtz coils that have unequal currents, producing MF gradients. The MF feedback probe is compensated (offset) for the injected ATPSI signals on each axis.

FIG. 8

Focused ATPSI setup neutralizing ambient dc and ac MFs using three orthogonal pairs of Helmholtz coils powered by a combined dc-ac source attenuating to very low values the dc and ac MFs. The injection of the undisturbed ATPSI field generating ATPSI conditions at a single point is through “gradient” Helmholtz coils added to the existing pair for each of the three axes, producing MF gradients. The MF feedback probe is compensated (offset) for the injected ATPSI signals on each axis. More coil additions can be envisioned along all axes to optimize or control the slope (steepness) of MF gradients.

### DESCRIPTION OF EMBODIMENTS FOR THE APPLICATION OF MF ATPSA AND ATPSI TO LIVING ORGANISMS

ATPSI and ATPSA can be implemented in living organisms both as acute and chronic treatments. Although the particulars of setups for treatments that last hours as opposed to months may differ in a number of practical details, they share some essential characteristics.

ATPSI and ATPSA can be used either as whole body or focused treatments, meaning that in the latter, the ATPSI and ATPSA conditions would be realized only within a limited spatial zone.

In some implementations, such as for the treatment of cancer, it is possible that patients would be primed under certain ATPSI-ATPSA conditions for periods of time in order to manipulate the metabolic state of the cells subsequently destroyed using other ATPSI-ATPSA conditions. It is also possible that pharmacological therapies could be used in conjunction with ATPSI-ATPSA to achieve therapeutic objectives.

### Whole Body ATPSA

The implementation of whole-body ATPSA is in principle the simplest, as it essentially involves a location within which both alternating and static MFs have been reduced to very small values. For maximum ATPSA, limits of 10 nT should be considered for both alternating and static MFs. We have realized these conditions in small volumes for the purpose of experimentation on living cells (Appendix 1). We achieved the proper MF attenuation in the center (3×3×3 cm) of an acrylic cylinder (5.7 cm in inner diameter, 0.38 cm wall and 38 cm long) covered by 6 layers of 0.4 mm NIM alloy foil (ASTM A753 Type 4) wound in a spiral, together with a 1.6 mm neoprene membrane spacer. The cylindrical shield was de-gaussed, and contained inside an incubator, which also provided some attenuation against static MFs.

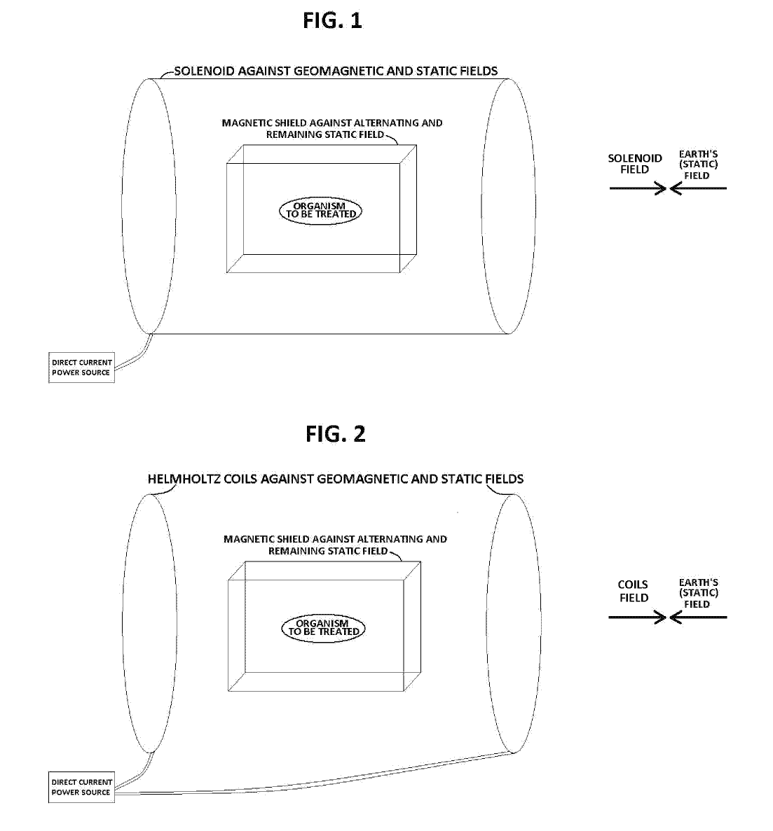

The implementation of ATPSA within a room or cavity of larger volume is straightforward, but quantitative considerations are important to control costs. If the maximum ATPSA condition is the target (attenuating both alternating and static fields to very low values), and as environmental alternating fields comparable in magnitude to the geomagnetic field (50 T) are rare, the limiting condition is the shielding of the Earth's MF. The reduction of 50 μT to 10 nT implies a MF attenuation by a factor of 5000. Dimensions of the shielded cavity will influence the cost of the installation, as for implementation of a shield as a long cylindrical shape, for example, the effective attenuation attained by the shielding layer is proportional to the magnetic permeability of the shielding material divided by its diameter. Because of the necessity of providing openings for access in enclosures of limited size, the effectiveness of the shielding usually falls far below theoretical calculations taking only magnetic permeability of the shielding material and geometry into account. This is particularly true if openings have to be included in relatively small enclosures for human access. The implication is that substantial amounts of money may need to be spent on shielding to realize extreme ATPSA conditions. For economic considerations, and assuming that adequate volumes with uniform MFs are available (no large ferro-magnetic materials in proximity) it may be preferable to place a more lightly shielded enclosure within a large solenoid (FIG. 1) that is powered by a direct current, and oriented with its axis parallel to the geomagnetic field in such a way that the geomagnetic field is mostly compensated by the current flowing through the solenoid, to the level of the local alternating field level (say, 0.1 μT) or less. A tight (many turns of wire per unit distance) solenoid produces an essentially constant magnetic flux within a good fraction of its volume near its center. Then, a shielded room within such a compensating solenoid faces requirements in shielding factor of only 100, which is more easily achievable at reasonable expense. The needed attenuation for the magnetic shield contained within the solenoid can then be obtained by a variety of enclosure shapes that use a small number of relatively thick layers of magnetically permeable material, which will be effective against both environmental alternating and remaining static fields. The solenoid can be build from any hollow solid structure allowing the winding of a varnished copper wire 0.45 mm in diameter (AWG 25), for example. The exact current needed in the solenoid to compensate the geomagnetic and other static fields depends on the number of wire turns.

When faced with the practical difficulty of building a large solenoid, an often-used alternative approach involves a number of Helmholtz-style coils (FIG. 2 shows a single pair of Helmholtz coils) which could have either circular, square or rectangular sections (to fit a room). The volumes within which specific MF conditions are met at given accuracies have been A4 described (Kirschvink, 1992). Helmholtz-style coils are usually installed in even numbers, and aligned coaxially around the volume to be magnetically controlled.

Rather than using a single pair of coils to substitute a single solenoid with its axis parallel to the Earth's MF as in FIG. 2, it may be practical to use 3 orthogonal rectangular pairs of coils, fitting the geometry of a room. Although such an arrangement is less costly and easier to build, it results in a smaller volume being compensated within a given MF precision than in the case of a solenoid. For most implementations, the necessary MF conditions would be successfully achieved for only a fraction of the shield volume near its center.

In order to protect the ATPSA environment from MF distortions or external MF parasites, the introduction into the chamber of magnetic materials or of MF radiating devices near the shielded room should be prevented. To monitor room performance, MF threshold probes should be placed strategically within the room. Such MF probes are available commercially, and use magnetoresistive or Hall effect probes for static fields, and induction coils for alternating MFs.

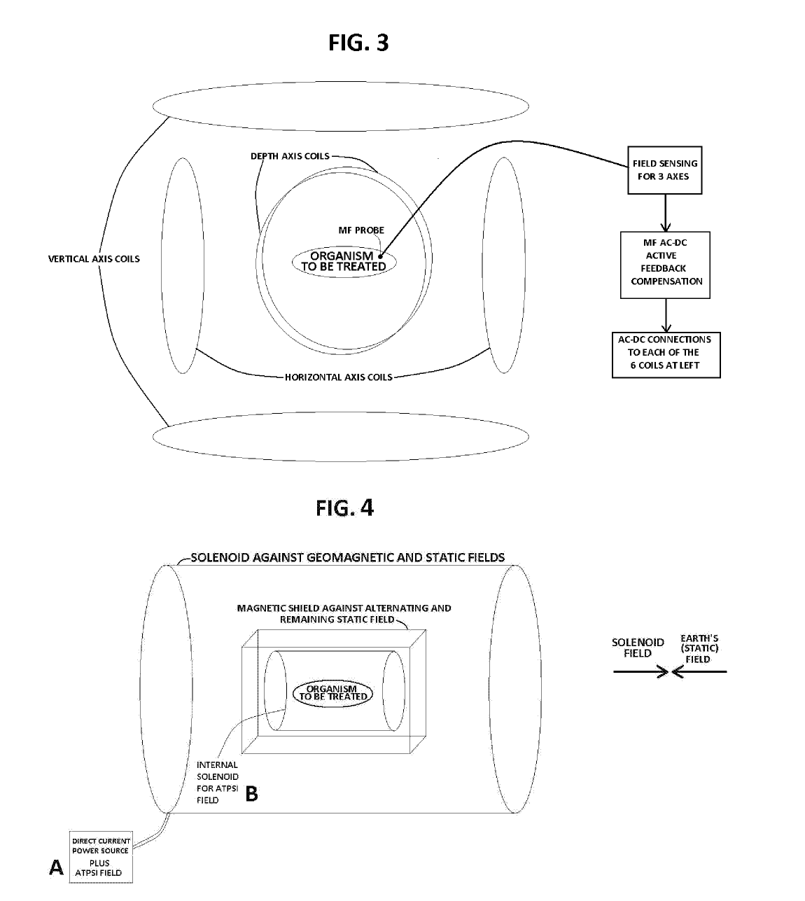

It is also possible to supply the right MF-depleted ATPSA environment by mere selection of a location that naturally provides such fields (away from electrical circuits and in a location where the Earth's field is naturally neutralized) or to create (FIG. 3) such conditions using arrays of coils and/or solenoids (typically three orthogonally oriented pairs), foregoing the use of shields entirely. In this implementation, arrays of coils are used to neutralize alternating, and possibly the static field as well, to very small values using the feedback from one or more probes sensing the magnetic environment in the volume to be treated. The probe's signals drive the proper counter-acting currents to compensation coils (active field compensation) that null the field within the required tolerance in the location of the organism under treatment.

### Whole Body ATPSI

Whole body ATPSI is a modification of the whole body ATPSA implementations described in FIGS. 1, 2 and 3 above. The technical requirement is to create a uniform alternating MF that fulfills the needed ATPSI conditions, optionally together with a static MF component. In its simplest form, the ATPSI condition is a MF sinewave at a particular combination of frequency and amplitude.

The ATPSA compensating solenoid and coils already described for whole body ATPSA in FIGS. 1, 2 and 3 above would be fed with not only the “Direct Current Power Source” signal of FIGS. 1 and 2 that neutralize the static field and with the “MF AC-DC Active Feedback Compensation” of FIG. 3 that neutralizes both alternating and static MFs, but with added current(s) that establish the proper ATPSI MF conditions inside the magnetic shield. This current could be applied either from outside the shield (point “A” in FIG. 4) or from within the shield by the addition of an extra solenoid and current signal (point “B” in FIG. 4)

In a similar way, FIG. 2 can be modified to FIG. 5, which includes “A” and “B”, alternate insertion points for the ATPSI signals.

Helmhotz-style coils and solenoids can fulfill similar roles in these embodiments, and the solenoids and coils can be implemented with arbitrary cross-sectional shapes.

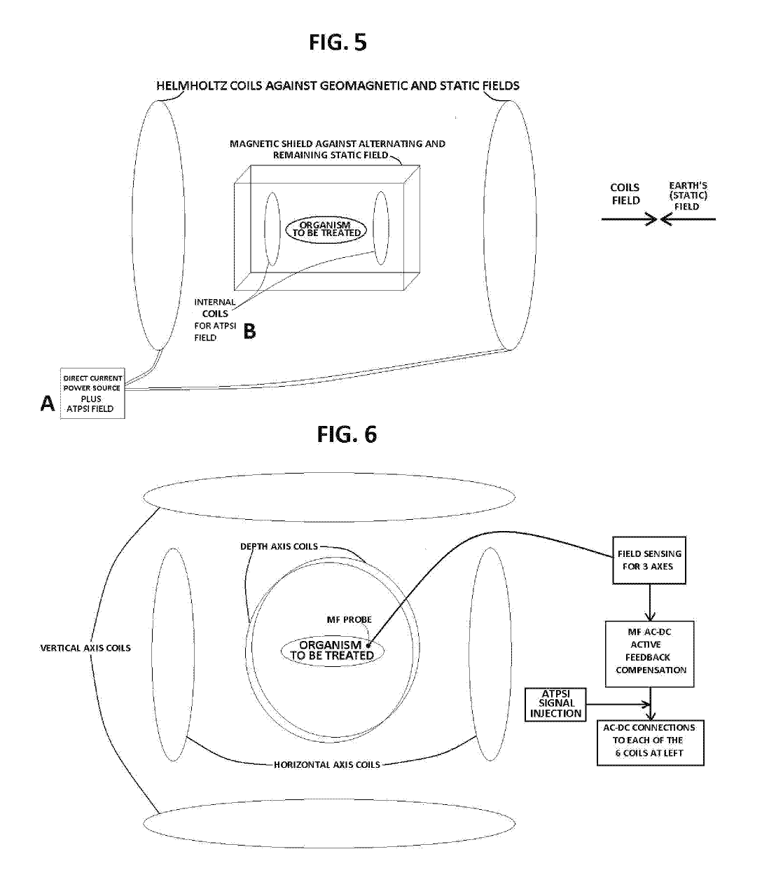

In the shield-less system of FIG. 3, the only need to transition from an ATPSA to an ATPSI configuration is addition of the “ATPSI Signal Injection” pictured in FIG. 6, properly compensated by a MF probe offset, which insures the realization of ATPSI conditions within the volume of the organism to be treated.

Considerable reductions in cost would be achieved if the needed ATPSI field only needed an alternating MF component, and could include the natural local geomagnetic field, as this would reduce drastically the amount of shielding efforts needed. In some cases, shielding may be eliminated altogether if the naturally-present geomagnetic field is tolerable, and if the ATPSI Helmholtz coils take into account any local MFs present at the site to establish ATPSI conditions, most likely with an adaptive electronic feedback system including a MF probe.

### Focused ATPSI

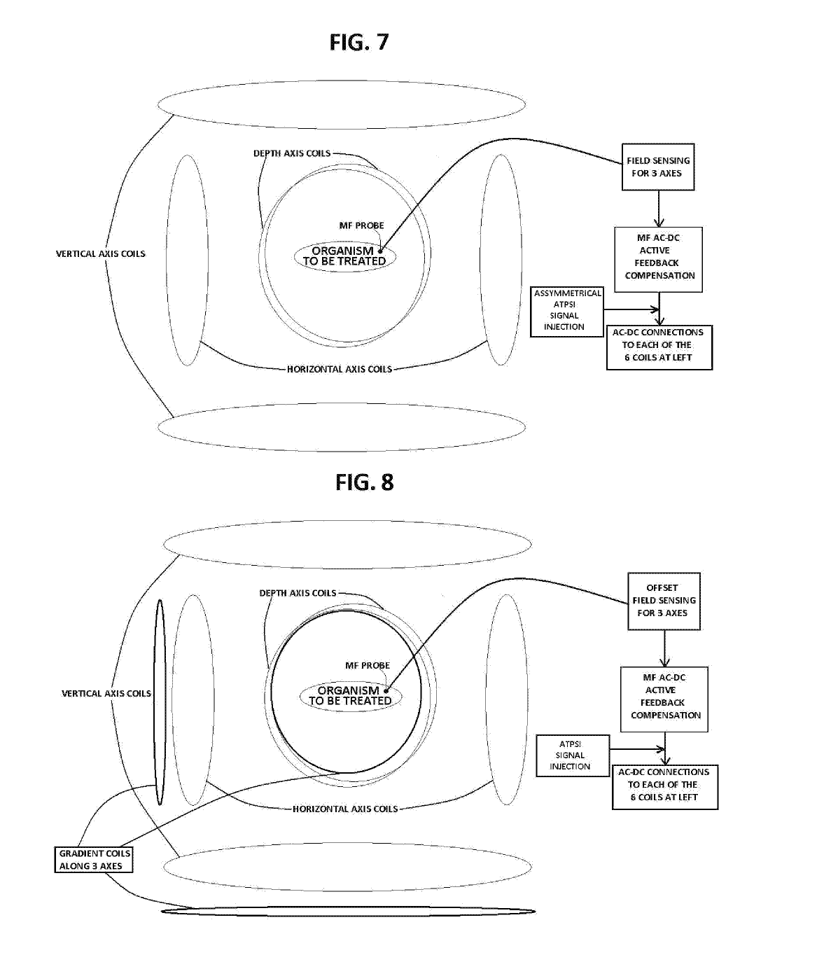

If it is desired to inhibit ATPS in a very specific location as opposed to a large volume, MF gradients are used. The appropriate alternating MF configuration can be understood by considering solenoids that, rather than being wound with a uniform pitch (turns per unit length) as in the whole body application of FIG. 4, are wound with a pitch that changes along their length. In FIG. 4, this would mean that either the “A” or the “B” solenoid, depending on which one is chosen for implementation, would be wound with a variable pitch, so that the solenoid produces a MF intensity that varies along with length. Specific ATPSI conditions would be closely met within a plane perpendicular to the solenoid axis. If, then, a second solenoid with a similar pitch gradient along its length and with its axis perpendicular to the first is added, this will result in the intersection of two planes whose combined fields fulfill the apex ATPSI condition, but at only one line (the intersection of the two planes). A third solenoid with similar configuration could restrict the ATPSI condition to a single point. Illustration of all three axes using solenoids would not be as clear as illustration using Helmholtz-like coils, which is shown in FIG. 7. Also, because of the encumbrance of fitting three solenoids within each other, focused ATPSI arrangements are more likely to be realized using arrays of Helmholtz-like coils. In FIG. 7, the modification for focused ATPSI is that the currents injected into the pairs of Helmholtz coils (for all axes) are deliberately made to have different values, which results in MF gradients along the three orthogonal directions, and consequently on control over the volume where effective ATPSI conditions are found. An alternative embodiment is shown in FIG. 8, where, instead of manipulating the currents of Helmholtz coil pairs, a “gradient” coil is added near one of the two Helmholtz coils for each axis, together with an offset signal on each axis of the nulling field sensing probe, to avoid feedback neutralization of the gradient coil signal. If one is further willing to add oppositely polarized steepness coils shadowing all Helmholtz coils, the steepness of the field decays, and so the volume of space in which strong ATPSI conditions are produced, could be varied.

In practice, depending on construction details, it may be possible to limit ATPSI conditions to within a small volume (rather than a point) by using fewer than three gradient solenoids or orthogonal coil sets, which would simplify construction.

The static MF condition, if needed within ATPSI, can be implemented by considering the need to establish a static MF of given value within a small volume co-localized with the alternating field conditions described above. The same Helmholtz-like coils in orthogonal configuration used above could pass a continuous current component that would add a static field to the ATPSI alternating field already generated.

### Focused ATPSA

If it is desired to enhance ATPS activity in a very specific location as opposed to a large volume, MF gradients are used, similar to what is described above. Starting from the whole body ATPSA condition described previously, pairs of oppositely polarized Helmholtz coils are installed whose MFs would neutralize at their center and at an equal distance between them. These coils could be used for both static and alternating fields, and the effective ATPSA volume could be reduced as described above by steepness coils. Three such arrangements orthogonal to each other combine to produce a single ATPSA point location. Smaller numbers of coils may be required for less precise confinement of ATPSA conditions.

### Chronic Application

In cases where chronic ATPSA or ATPSI treatments are necessary, it may be desirable to design ambulatory exposure systems which could be used within a shielded room(s), but would not restrict patient mobility. In ATPSA, the shielded room itself may be sufficient, assuming that the patient is restricted to the properly compensated volume.

For the various ATPSI strategies, solenoids inserted around body parts could be an effective implementation. Depending upon the precision with which ATPSI conditions need to be realized for various applications, it may be possible to implement completely ambulatory systems that a patient would “wear”. This would be particularly practical if static fields were ignored as unimportant or acceptable for a given application, and if an active compensation system that altered the field supplied by the ATPSI coil according to a feedback loop from a magnetic sensor located near the treatment volume was available. In this implementation, the magnetic sensor is the controlling system driving the current intensity within the magnetic coil to insure a pre-determined ATPSI condition within the treated volume.

In a highly sophisticated implementation, ATPSA and ATPSI systems could be combined with Nuclear Magnetic Resonance scanners set to localize the position in space of malignant cells. In this case, a coordinated ATPSI system could automatically adjust to provide the appropriate ATPSI conditions at those coordinates, thereby relaxing the immobility requirements placed on patients.

### Magnetic ATPS Control Applied to Other Diseases and Conditions

Beyond applications in cancer therapy, which require strong ATPS, there may be a number of medical conditions for which control of cellular respiration, and therefore ATPS control, may be desirable. These applications could involve algorithms for weak or strong ATPSI or ATPSA exposures over time. Complex MF procedures may be useful to deal with chronic diseases, and treatments could even be applied during sleep, for example. Possible applications of ATPSI or ATPSA would include antibiotic effect, Alzheimer's disease, presenile dementia, autoimmune disorders (lupus erythematosus), obesity, antiangiogenic cancer therapy (Hong and Pedersen, 2008), and it could be developed as a method to deal with insomnia. As ATPSI suppresses cellular respiration, much as hydrogen sulphide does, it may be applied to situations such as anaesthesia and suspended animation.

### Description of Embodiments for the Application of MF ATPSA and ATPSI to Fluids

Because fluids and other water-containing substances can usually be packaged into small containers, implementation of ATPSA-ATPSI is more simple and inexpensive than for large living organisms. For example, if the ATPSA requirement is to shield a packaged fluid from environmental alternating MFs only, an inexpensive and entirely passive implementation would consist of a thick steel cylinder (6.5 cm internal diameter, 1.3 cm wall, 45 cm long) within which a bottle of fluid (350 ml) is centered. Keeping in mind that about 4 hours are necessary for the water ordering allowing ATPSA to take hold in the fluid, and that the effect will dissipate within 2 hours, such a pipe could “produce” ATPSA fluids at intervals. If the ATPSA condition required includes both low alternating and low geomagnetic fields, the shield becomes more complex as it needs to neutralize both types of MFs. In this case, more specialized shielding made of the NIM foil previously mentioned, for example, would be needed. To maintain very high shielding factors over time, such shields would probably need to be de-gaussed periodically with a decaying alternating current passing through the inside of the shield, as they can be re-magnetized over time as a result of environmental conditions.

If ATPSI fluids are needed, the system described above can provide a low MF background, and a solenoid imbedded within the shield can supply the means to apply the needed ATPSI condition to the fluid, using an external power source that could include both continuous and alternating currents.

### PREVIOUS ART

Previous art has been published on the control of ATPS (Group A below), primarily for cancer therapy, but all of theses patents involve molecules rather than fields as the therapeutic agent. Previous art has been published on the use of MFs as cancer therapies (Group B below), but none of these techniques involve ATPS or mention water structure. A distinguishing feature is that the present invention is focussed on the control of a specific mitochondrial enzyme, ATPS. Some comments are given on selected patents of Group B in tabular form below.

Group C contains previous art on MF methods for treating water. All use very strong static MFs using magnets, most of them for the purpose of controlling or eliminating scale build-up from water pipes. All are flow-through devices, in contrast to our apparatus, which treats water over hours to achieve any efficiency. Only a few use solenoids to provide uniform static or alternating fields. None of the previous art involve shielding water from extraneous MF influences to achieve exact control over exposure. Hatton mentions “living water” and Rodriguez “revitalizing water”, obviously implying that the treatment will produce a stimulating physiological effect. This is quite opposite to our apparatus, which would induce an increase in cellular respiration by eliminating MFs.

Pilla (2009) stands out from this group by describing a specific combination of magnets to achieve therapeutic-type effects on living materials, based on Larmor resonance and the calcium ion.

TABLE 2
Comments on Prior Art

| | |
|---|---|
| Gordon, | Talks of “electromagnetic energy”, interferon, and the existence of a |
| 1986 | “resonant frequency” of normal and cancer cells, both of which have no |
| | factual basis. |
| Costa, | Magnetic pulses of intensities between 1 and 100 T and between 5 and 1000 kHz. |
| 1987 | |
| Mills, | Uses Moosbauer absorption: gamma radiation. |
| 1989 | |
| Liboff, | A cyclotron resonance proposal, in which the method attempts to influence |
| 1991 | the movement of specific ions within cells according to their gyromagnetic |
| | ratio. This supposes that the ion potassium (and others) will obey the |
| | magnetic field at a certain combination of frequencies and field intensities. |
| | This hypothesis as not been confirmed by experiments. |
| Liboff, | Based on the same principles as Liboff, 1991. |
| 1993 | |
| Litovitz, | This is method aims to avoid the health effects of magnetic fields by |
| 1995 | introducing fluctuations in otherwise regular sinusoid oscillations. |
| Bonlie, | The therapy is a static field with at least 500 Gauss intensity. That assumes |
| 2001 | that cells originally divided in static fields that were too weak, and need more |
| | static field exposure. |
| Litovitz, | A re-edition of the method of introducing fluctuations in magnetic fields to |
| 2001 | thwart their nefarious effects. |
| Blackman, | The ion parametric resonance, an upgrade of the cyclotron resonance, that |
| 2001 | uses both static and alternating field to influence the behavior of small ions. |
| Davey, | Uses electric fields to alter the protein p53 and regenerate nerves. |
| 2002 | |
| Litovitz, | Electromagnetic fields between 5 Hz and 5 GHz are modulated at 10 second |
| 2005 | intervals to “cure” a wide variety of diseases through the control of heat shock |
| | proteins. |
| Litovitz, | Alternatingly active magnetic fields are used to induce electric fields in the |
| 2009 | living body. The electric field interacts with membranes, and supposedly |
| | creates therapeutic effects. |
| Vasista, | Uses a programmed sequence of pulsing magnetic fields to induce electric |
| 2010 | fields which either regenerate or impair survival of organisms. |
| Sivo, | A 10 MHz bipolar sinusoidal, square, triangular or sawtooth currents in bursts |
| 2010 | and pulses (1.5 or 2 μseconds in duration) and modulated at about 15 Hz or |
| | 200 kHz that claim to induce arrest of the growth or destroy cancers. The |
| | proposal claims an electromagnetic field of peak amplitude between 1 and 300 V/m. |

### CITATION LIST

### Patent Literature

### Group A (US Patents)

US Patent Application 2010/0190845 A1. Date: Jul. 29, 2010. METHOD OF TREATING CANCER USING ATP SYNTHASE INHIBITORS. Juan et al.
US Patent Application 2003/0086929 A1. Date: May 8, 2003. TREATMENT OF PROSTATE CANCER BY INHIBITORS OF ATP SYNTHASE. Tso et al.
US Patent Application 2003/0026781A1. Date: Feb. 6, 2003. COMPOSITIONS AND METHODS FOR REGULATING ENDOGENOUS INHIBITOR OF ATP SYNTHASE, INCLUDING TREATMENT FOR DIABETES. Anderson et al.
U.S. Pat. No. 6,376,211 B1. Date: Apr. 23, 2002. AGENTS AND METHODS FOR INHIBITING F1/F0 ATPASE. Little, I I et al.
US Patent Application 2007/0203083. Date: Aug. 30, 2007. METHODS OF REGULATING METABOLISM AND MITOCHONDRIAL FUNCTION. Mootha et al.
US Patent Application 2011/0110914 A1. Date: May 12, 2011. METHODS FOR TREATMENT OF ONCOLOGICAL DISORDERS USING EPIMETABOLIC SHIFTERS, MULTIDIMENSIONAL INTRACELLULAR MOLECULES, OR ENVIRONMENTAL INFLUENCERS. Narain et al.

### Group B (US Patents)

U.S. Pat. No. 4,622,952. Date: Nov. 18, 1986. CANCER TREATMENT METHOD. Gordon.
U.S. Pat. No. 4,665,898. Date: May 19, 1987. MALIGNANCY TREATMENT. Costa.
U.S. Pat. No. 4,815,448. Date: Mar. 28, 1989. MOSSBAUER CANCER THERAPY. Mills.
U.S. Pat. No. 5,045,050. Date: Sep. 3, 1991. METHOD AND APPARATUS FOR THE TREATMENT OF CANCER. Liboff.
U.S. Pat. No. 5,211,622. Date: May 18, 1993. METHOD AND APPARATUS FOR THE TREATMENT OF CANCER. Liboff.
U.S. Pat. No. 5,450,859. Date: Sep. 19, 1995. PROTECTION OF LIVING SYSTEMS FROM ADVERSE EFFECTS OF ELECTRIC, MAGNETIC AND ELECTROMAGNETIC FIELDS. Litovitz.
U.S. Pat. No. 6,210,317 B1. Date: Apr. 3, 2001. TREATMENT USING ORIENTED UNIDIRECTIONAL DC MAGNETIC FIELD. Bonlie.
U.S. Pat. No. 6,263,878 B1. Date: Jul. 24, 2001. MEANS FOR PROTECTING LIVING SYSTEMS FROM ADVERSE EFFECTS OF ELECTRIC, MAGNETIC AND ELECTROMAGNETIC FIELDS. Litovitz.
U.S. Pat. No. 6,238,899 B1. Date: May 29, 2001. METHOD AND APPARATUS FOR ALTERING IONIC INTERACTIONS WITH MAGNETIC FIELDS. Blackman.
US Patent Application 2002/0103515 A1. Date: Aug. 1, 2002. MAGNETIC FIELDS FOR THE TREATMENT OF CANCER AND TO ASSIST 1N NERVE REGENERATION. Davey.
U.S. Pat. No. 6,856,839 B2. Date: Feb. 15, 2005. USE OF ELECTROMAGNETIC FIELDS IN CANCER AND OTHER THERAPIES. Litovitz.
U.S. Pat. No. 7,587,230 B2. Date: Sep. 8, 2009. METHOD OF USING MAGNETIC FIELDS TO UNIFORMLY INDUCE ELECTRIC FIELDS FOR THERAPEUTIC PURPOSES. Litovitz.
US Patent 2010/0197993 A1. Date: Aug. 5, 2010. SEQUENTIALLY PROGRAMMED MAGNETIC FIELD THERAPEUTIC SYSTEM (SPMF). Vasishta.
US Patent Application 2010/0016651A1. Date: Jan. 21, 2010. METHODS TO ARREST CANCER CELL GROWTH AND PROLIFERATION USING ELECTROMAGNETIC ENERGY DELIVERED VIA ELECTROMAGNETIC COIL SYSTEMS. Sivo.

### Group C (US Patents)

US Patent Application 2009/0071889 A1. Date: Mar. 19, 2009. MAGNETIC FIELD GENERATING DEVICE FOR POLARIZING FLUIDS AND DISTRIBUTION COMPONENTS USING THE SAME. Arenas et al.
U.S. Pat. No. 4,146,479. Date: Mar. 27, 1979. MAGNETIC WATER CONDITIONER. Brown.
U.S. Pat. No. 5,296,141. Date: Mar. 22, 1994. MAGNETIC WATER CONDITIONER. Ellison.
U.S. Pat. No. 6,797,159 B2. Date: Sep. 28, 2004. APPARATUS FOR RECIRCULATING VORTEX WATER FOUNTAIN. Hatton.
US Patent Application 2005/0126974 A1. Date: Jun. 16, 2005. WATER PURIFIER HAVING MAGNETIC FIELD GENERATION. Naito.
U.S. Pat. No. 6,171,504 B1. Date: Jan. 9, 2001. MAGNETIC WATER CONDITIONER. Patterson.
US Patent Application 2009/0306456 A1. Date: Dec. 10, 2009. APPARATUS AND METHOD FOR STATIC MAGNETIC FIELD TREATMENT OF TISSUE, ORGANS, CELLS, AND MOLECULES. Pilla et al.
U.S. Pat. No. 5,288,401. Date: Feb. 22, 1994. MAGNETIC INDUCTOR TO REVITALIZE WATER. Rodriguez.
U.S. Pat. No. 3,951,807. Date: Apr. 20, 1976. WATER CONDITIONING APPARATUS. Sanderson.
US Patent Application 2009/0242407 A1. Date: Oct. 1, 2009. ELECTROMAGNETIC FIELD TREATMENT METHOD AND ELECTROMAGNETIC FIELD TREATMENT EQUIPMENT OF WATER. Shiga.
US Patent Application 2008/0277352 A1. Date: Nov. 13, 2008. MAGNETIC WATER CONDITIONER. White.
US Patent Application 2009/0261028 A1. Date: Oct. 22, 2009. MAGNETIC FIELD FORMING DEVICE FOR ACTIVE WATER AND FLUID TREATMENT APPARATUS USING THE SAME. Yamasaki.
U.S. Pat. No. 7,943,043 B2. Date: May 17, 2011. MAGNETIC FIELD FORMING DEVICE FOR ACTIVE WATER AND FLUID TREATMENT APPARATUS USING THE SAME. Yamasaki.
U.S. Pat. No. 6,602,411 B1. Date: Aug. 5, 2003. MAGNETIC TREATING APPARATUS OF WATER. Aida et al.

### Non Patent Literature

Bonnet Sébastien, Stephen L. Archer, Joan Allalunis-Turner, Alois Haromy, Christian Beaulieu et al. January 2007. A Mitochondria-K Channel Axis Is Suppressed in Cancer and Its Normalization Promotes Apoptosis and Inhibits Cancer Growth. Cancer Cell 11, 37-51.
Edwards Steven W. and David Lloyd. 1977. Mitochondrial Adenosine Triphosphatase of the Fission Yeast. Schizosaccharomyces pombe 972h-. Changes in Activity and Oligomycin-Sensitivity during the Cell Cycle of Catabolite-Repressed and -De-Repressed Cells. Biochem. J. 162, 39-46.
Hong Sangjin and Peter L. Pedersen. December 2008. ATP Synthase and the Actions of Inhibitors Utilized to Study its Roles in Human Health, Disease, and Other Scientific Areas. Microbiology and Molecular Biology Reviews, p. 590-641, Vol. 72, No. 4. doi:10.1128/MMBR.00016-08.
Kirschvink Joseph L. 1992. Uniform Magnetic Fields and Double-Wrapped Coil Systems: Improved Techniques fbr the Design of Bioelectromagnetic Experiments. Bioelectromagnetics 13:401-411.
Korshunov Sergey S., Vladimir P. Skulachev, Anatoly A. Starkov. 1997. High protonic potential actuates a mechanism of production of reactive oxygen species in mitochondria FEBS 19307 FEBS Letters 416 (1997) 15-18.
Lardy Henry A., Philip Witonsky, and Diane Johnson. Antibiotics as Tools for Metabolic Studies. 1965. IV. Comparative Effectiveness of Oligomycins A, B, C, and Rutamycin as Inhibitors of Phosphoryl Transfer Reactions in Mitochondria. Biochemistry vol. 4, no. 3, March.
Li Y, Héroux P and Kyrychenko I. 2012. Metabolic Restriction of Cancer Cells in vitro causes Karyotype Contraction—an indicator of Cancer Promotion? 2012. Tumor Biology, 33(1):195-205 DOI:10.1007/s13277-011-0262-6.
Miller Daniel J., Norbert A. Dencher, Thomas Meier, Peter Dimroth, Kitaru Suda, Henning Stahlberg, Andreas Engel, Holger Seelert, Ulrich Matthey. 2001. ATP synthase: constrained stoichiometry of the transmembrane rotor. FEBS Letters 504 219-222.
Reed John C. 2011. Priming Cancer Cells for Death. November 2011. Science Vol 334, 25, pp. 1075-6.
Reiter G F, Kolesnikov A I, Paddison S J, Platzman P M, Moravsky A P et al. 2011. Evidence of a new quantum state of nano-confined water. arXiv: 1101.4994v1.
Salomon Arthur R., David W. Voehringert, Leonard A. Herzenbergt, and Chaitan Khosla. Dec. 19, 2000. Understanding and exploiting the mechanistic basis for selectivity of polyketide inhibitors of FOF1-ATPase. PNAS I, vol. 97, no. 26.
Salomon Arthur R., David W. Voehringer, Leonard A. Herzenberg, Chaitan Khosla. 2001. Apoptolidin, a selective cytotoxic agent, is an inhibitor of F0 F1-ATPase. Chemistry & Biology 8 71-80.
Samudio Ismael, Michael Fiegl, and Michael Andreeff. Mar. 15, 2009. Mitochondrial Uncoupling and the Warburg Effect: Molecular Basis for the Reprogramming of Cancer Cell Metabolism. Cancer Res 69: (6). Published OnlineFirst Mar. 3, 2009; DOI: 10.1158/0008-5472.CAN-08-3722.
Semenza Gregg L. 1999. Regulation of Mammalian O2 Homeostasis by Hypoxia-Inducible Factor 1 Annu. Rev. Cell Dev. Biol. 15:551-78.
Semikhina, L P, Kiselev V F. 1981. Effect of weak magnetic fields on the properties of water and ice. Russian Physics Journal 31:5351-54.
Semikhina L P, Kiselev V F, Levshin L V, Saletskii A M. 1988. Effect of weak magnetic fields on the luminescence-spectral properties of a dye in an aqueous solution. Journal of Applied Spectroscopy 48:556-9.
Skulachev Vladimir P., 1996. Role of uncoupled and non-coupled oxidations in maintenance of safely low levels of oxygen and its one-electron reductants. Quarterly Reviews of Biophysics, 29: pp 169-202. doi: 10.1017/S0033583500005795.
van der Windt Gerritje J. W., Bart Everts, Chih-Hao Chang, Jonathan D. Curtis, Tori C. Freitas, Eyal Amiel, Edward J. Pearce, and Erika L. Pearce. Jan. 27, 2012. Mitochondrial Respiratory Capacity is a Critical Regulator of CD8+ T Cell Memory Development. Immunity 36, 68-78. doi 10.1016/j.immuni.2011.12.007.
Warburg O. On the origin of cancer cells. Science. 1956. 123(3191):309-14.

1. Use of the elimination of magnetic field exposures of living organisms to enhance the function of the enzyme adenosine triphosphate synthase.

2. Use of the elimination of static and ELF magnetic field exposures of living organisms to enhance the function of the enzyme adenosine triphosphate synthase.

3. Use of the elimination of magnetic fields exposures of living organisms to enhance the function of the enzyme adenosine triphosphate synthase as a treatment for cancer.

4. Use of the elimination of static and ELF magnetic fields exposures of living organisms to enhance the function of the enzyme adenosine triphosphate synthase as a treatment for cancer.

5. Use of the elimination of magnetic field exposures of living organisms to enhance the function of the enzyme adenosine triphosphate synthase as a treatment for human diseases.

6. Use of the elimination of static and ELF magnetic field exposures of living organisms to enhance the function of the enzyme adenosine triphosphate synthase as a treatment for human diseases.

7. Use of magnetic fields applied to living organisms to control the efficiency of the enzyme adenosine triphosphate synthase.

8. Use of static and ELF magnetic fields applied to living organisms to control the efficiency of the enzyme adenosine triphosphate synthase.

9. Use of magnetic fields applied to living organisms to control the efficiency of the enzyme adenosine triphosphate synthase as a treatment for cancer.

10. Use of static and ELF magnetic fields applied to living organisms to control the efficiency of the enzyme adenosine triphosphate synthase as a treatment for cancer.

11. Use of magnetic fields applied to living organisms to control the efficiency of the enzyme adenosine triphosphate synthase as a treatment for human diseases.

12. Use of static and ELF magnetic fields applied to living organisms to control the efficiency of the enzyme adenosine triphosphate synthase as a treatment for human diseases.

13. Use of magnetic field transitions applied to living organisms to induce adenosine triphosphate synthase efficiency changes.

14. Use of static and ELF magnetic field transitions applied to living organisms to induce adenosine triphosphate synthase efficiency changes.

15. Use of magnetic field transitions applied to living organisms to induce adenosine triphosphate synthase efficiency changes as a treatment for cancer.

16. Use of static and ELF magnetic field transitions applied to living organisms to induce adenosine triphosphate synthase efficiency changes as a treatment for cancer.

17. Use of magnetic field transitions applied to living organisms to induce adenosine triphosphate synthase efficiency changes as a treatment for human diseases.

18. Use of static and ELF magnetic field transitions applied to living organisms to induce adenosine triphosphate synthase efficiency changes as a treatment for human diseases.

19. Use of water-containing liquids or substances previously shielded from magnetic fields to enhance the efficiency of the enzyme adenosine triphosphate synthase within living organisms, after injection or absorption of such water-containing liquids or substances.

20. Use of water-containing liquids or substances previously shielded from static and ELF magnetic fields to enhance the efficiency of the enzyme adenosine triphosphate synthase within living organisms, after injection or absorption of such water-containing liquids or substances.

21. Use of water-containing liquids or substances previously shielded from magnetic fields to enhance the efficiency of the enzyme adenosine triphosphate synthase within living organisms, after injection or absorption of such water-containing liquids or substances as a treatment for cancer.

22. Use of water-containing liquids or substances previously shielded from static and ELF magnetic fields to enhance the efficiency of the enzyme adenosine triphosphate synthase within living organisms, after injection or absorption of such water-containing liquids or substances as a treatment for cancer.

23. Use of water-containing liquids or substances previously shielded from magnetic fields to enhance the efficiency of the enzyme adenosine triphosphate synthase within living organisms, after injection or absorption of such water-containing liquids or substances as a treatment for human diseases.

24. Use of water-containing liquids or substances previously shielded from static and ELF magnetic fields to enhance the efficiency of the enzyme adenosine triphosphate synthase within living organisms, after injection or absorption of such water-containing liquids or substances as a treatment for human diseases.

25. Use of water-containing liquids or substances previously exposed to magnetic fields to control the efficiency of the enzyme adenosine triphosphate synthase within living organisms, after injection or absorption of such water-containing liquids or substances.

26. Use of water-containing liquids or substances previously exposed to static and ELF magnetic fields to control the efficiency of the enzyme adenosine triphosphate synthase within living organisms, after injection or absorption of such water-containing liquids or substances.

27. Use of water-containing liquids or substances previously exposed to magnetic fields to control the efficiency of the enzyme adenosine triphosphate synthase within living organisms, after injection or absorption of such water-containing liquids or substances as a treatment for cancer.

28. Use of water-containing liquids or substances previously exposed to static and ELF magnetic fields to control the efficiency of the enzyme adenosine triphosphate synthase within living organisms, after injection or absorption of such water-containing liquids or substances as a treatment for cancer.

29. Use of water-containing liquids or substances previously exposed to magnetic fields to control the efficiency of the enzyme adenosine triphosphate synthase within living organisms, after injection or absorption of such water-containing liquids or substances as a treatment for human diseases.

30. Use of water-containing liquids or substances previously exposed to static and ELF magnetic fields to control the efficiency of the enzyme adenosine triphosphate synthase within living organisms, after injection or absorption of such water-containing liquids or substances as a treatment for human diseases.
